(19) United States
(12) Patent Application Publication
(10) Pub. No.: US 2009/0081275 A1
(43) Pub. Date: Mar. 26, 2009

(54) Durable swellable hydrogel matrix and methods

(76) Inventors: Emily R. Rolfes, Eden Priarie, MN (US); Stephen J. Chudzik, St. Paul, MN (US); Pamela J. Reed, St. Paul, MN (US)

Correspondence Address: Kagan Binder, PLLC, 221 Main Street North, Suite 200, Stillwater, MN, 55082, US

(21) Appl. No.: 12/284,838

(22) Filed: Sep. 25, 2008

Related U.S. Application Data
(60) Provisional application No. 60/995,170, filed on Sep. 25, 2007.

(51) Int. Cl. A61F 2/04; A61K 9/10

(52) U.S. Cl. 424/423; 424/484; 424/486; 424/487

(57) ABSTRACT

The invention provides biocompatible polymeric hydrogel matrices having excellent durability and swellability. The matrices are formed from a combination of linear and branched hydrophilic macromer compounds. The matrices can be used in association with a medical device or alone. In some methods the polymeric matrix is placed or formed at a target site in which the matrix swells and occludes the target area.

### CROSS-REFERENCE TO RELATED APPLICATION

This application claims the benefit of U.S. Provisional Patent Application Ser. No. 60/995,170, filed Sep. 25, 2007, entitled DURABLE SWELLABLE HYDROGEL MATRIX AND METHODS, the disclosure of which is incorporated herein by reference.

### FIELD OF THE INVENTION

The invention is directed to hydrogels, and compositions and methods for their preparation. The invention also relates to systems and methods for the occlusion of an internal portion of the body by an implanted or formed article.

### BACKGROUND

A hydrogel is typically thought of as an insoluble matrix of crosslinked hydrophilic polymers having the capacity to absorb large amounts of water. Due to their physical properties and ability to be prepared from biocompatible materials, hydrogels have considerable use in biomedical applications. For example, hydrogels have been used as material for the treatment of wounds, as well as vehicles for the release of drugs. Hydrogels have also been used as coatings on the surface of medical devices, and can be used to improve the hydrophilicity or lubricity of the device surface.

Hydrogels are typically characterized by their capacity to swell upon absorption of water from a dehydrated state. This swelling can be affected by conditions in which the hydrogel is placed, such as by pH, temperature, and the local ion concentration and type. Several parameters can be used to define or characterize hydrogels in a swollen state, including the swelling ratio under changing conditions, the permeability coefficient of certain solutes, and the mechanical behavior of the hydrogel under conditions of its intended use.

Hydrogels that undergo a considerable degree of swelling can be useful for many medical applications in the body in which the hydrogel is placed, or is formed. However, hydrogels having a high degree of swelling may also be structurally unsuitable for use in the body. For example, considerable swelling may cause the hydrogel to become fragile, and fracture or fragment upon contact with body tissue. This could cause the hydrogel, or a device associated with the hydrogel, to lose its functionality, or could introduce complications in the body if a portion of the hydrogel is dislodged from the target site.

### SUMMARY

The present invention provides polymeric matrix-forming formulations, swellable polymeric matrices, medical articles associated with the swellable polymeric matrices, and methods of using the swellable polymeric matrices. The polymeric matrices of the invention are substantially swellable in aqueous environments to form hydrogels that are durable and well suited for use in the body. The swellable polymeric matrices are formed from a combination of polymeric materials that provide high water absorbing capacity as well as a high density of crosslinking. As such, the present invention addresses issues with swellable polymeric matrices that may demonstrate good swelling but result in hydrogels having insufficient structural properties, such as insufficient durability.

The swellable polymeric matrices of the invention are particularly useful when implanted or formed at a target site in the body. The polymeric matrices form swollen hydrogels that occlude a target area of the body, and provide a desired biological effect at the target site. The swellable polymeric matrices can be delivered to the target area in a dry, or partially dry (dehydrated) state, where at the target area, the matrices become hydrated and swell to occlude or block the area. The occlusion or blockage can have a biological effect. For example, the occluding hydrogel can prevent the movement of biological fluids, tissue, or other biological material, across or into the occluded area.

The polymeric matrices of the present invention provide the advantage of forming swollen hydrogels with improved durability, without loss of swellability. The use of the swellable polymeric matrices as described herein can therefore provide improved function in vivo. For example, the polymeric matrices are less likely to fracture following swelling. This can provide more complete occlusion or blockage at the target area and can also increase functional lifetime of the hydrogel following implantation.

The polymeric matrices can be used alone at the target area, or can be used in association with a device. For example, in some aspects, the polymeric matrices can be in the form of an overmold, or in the form of a coating on an implantable medical device. The non-hydrogel portion of the device can facilitate delivery and function of the hydrogel at the target site.

In one aspect, the invention provides a polymeric matrix-forming composition. The composition includes a linear hydrophilic polymer comprising a pendent reactive group, and a non-linear or branched compound comprising two or more hydrophilic polymeric portions and pendent reactive groups. The reactive groups on the linear hydrophilic polymer and the branched compound can be reacted to form a biocompatible polymeric matrix that can be substantially swollen to a durable hydrogel. The combination of these two matrix-forming components is thought to provide a polymeric matrix with a particular crosslinked architecture having excellent swellability and durability.

The linear hydrophilic polymer can be a oxyalkylene polymer, such as poly(ethylene glycol). The non-linear or branched compound can derived from a polyol. Exemplary polyol derivatives include oxyalkylene derivatives of pentaerythritol, trimethylolpropane, and glycerol.

The reactive groups can be polymerizable groups, and the hydrogel can be formed using a polymerization initiator.

In another aspect, the invention provides a swellable polymeric matrix formed of a crosslinked network of polymeric material comprising first and second polymer-containing segments. The crosslinked network comprises a first polymer-containing segment having a linear structure comprising a hydrophilic polymer portion, and a second polymer-containing segment having a non-linear or branched structure comprising hydrophilic polymeric portions, such as oxyalkylene polymer portions. The swellable polymeric matrix is substantially swellable and provides a durable hydrogel. In some formations, the polymeric matrix is capable of swelling in water to a weight in the range of 1.5 to 10 times its weight in a dehydrated form. In some formations is matrix is capable of exerting a swelling force in the range of 100 g/cm² to 2000 g/cm² upon hydration from a dehydrated form. In some formations, the polymeric matrix is capable of swelling in water to a size in the range of about 150% to about 300% its size in a dehydrated form.

In another aspect, the invention provides a medical device having a swellable polymeric matrix formed of a crosslinked network of polymeric material. The matrix can be associated with the device in various ways, such as in the form of an overmold or a coating on the device. The matrix comprises a first polymer-containing segment having a linear structure comprising a hydrophilic polymer, and a second polymer-containing segment having a non-linear or branched structure comprising hydrophilic polymeric portions, such as oxyalkylene polymer portions. The first and second polymer-containing segments are crosslinked via polymerized groups pendent from the polymer portions. The matrix is substantially swellable and provides a durable hydrogel upon swelling. The medical device can be configured for placement in target areas of the body, such as in aneurysms, and portions the reproductive tract, such as the fallopian tube. The medical device can be implanted in the body when the matrix is in a dehydrated form, and during and/or following implantation, the matrix can become rehydrated and swell. In some cases, the matrix is swellable upon placement in the body to provide the medical device with a diameter that is three times, or greater than three times, than the diameter of the device in the dehydrated form.

In another aspect, the invention provides a method for space filling or occluding an area within the body. The method includes a step of implanting an article comprising a swellable polymeric matrix, or forming a matrix at a target location in the body, the matrix formed of a crosslinked network comprising (i) a a first polymer-containing segment having a linear structure comprising a hydrophilic polymer, and (ii) a second polymer-containing segment having a non-linear or branched structure comprising hydrophilic polymeric portions. The method also includes a step of allowing the matrix to swell at the target site to form a hydrogel and occlude the target location in the body.

### DETAILED DESCRIPTION

The embodiments of the present invention described below are not intended to be exhaustive or to limit the invention to the precise forms disclosed in the following detailed description. Rather, the embodiments are chosen and described so that others skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

The present invention provides improved polymeric matrices that can be swollen in situ to a durable hydrogel that blocks or occludes a target area in the body. The swellable polymeric matrices are formed from two different polymeric-based components that have pendent reactive groups. The pendent reactive groups can be activated or reacted to crosslink the components to form a swellable polymeric matrix. Other components can optionally be included for formation of the matrix.

The swellable polymeric matrix can be used in various forms. For example, the swellable polymeric matrix can be in a form of an overmold on a medical device. The matrix can also be in the form of a coating on a medical device. The swellable polymeric matrix can also itself be used as a device itself (i.e., formed by the matrix-forming composition). The matrix can also be formed in situ at the target site.

One component (i.e., the first component) that is used to form the swellable polymeric matrix is a linear hydrophilic polymer comprising one or more pendent reactive groups. Another component (i.e., the second component) is a branched compound comprising two or more hydrophilic polymeric portions and pendent reactive groups. The reactive groups on the linear hydrophilic polymer and the branched compound can be reacted to form a polymeric matrix that is swellable to a durable hydrogel.

A “swellable polymeric matrix” refers to a crosslinked matrix of polymeric material formed from at least the first and second components. The polymeric matrix can either be dehydrated or can contain an amount of water that is less than the amount of water present in a fully swollen matrix (the fully hydrated matrix being referred to herein as a “hydrogel”). Typically, the matrix is not fully hydrated when delivered to a target site in the body for occlusion. The invention contemplates the matrix in various levels of hydration.

To facilitate discussion of the invention, polymerizable groups will be discussed as the reactive groups pendent from the components that form the swellable polymeric matrix. The linear hydrophilic polymer (i.e., a macromer) includes one or more “polymerizable group(s)” which generally refers to a chemical group that is polymerizable in the presence of free radicals. Polymerizable groups generally include a carbon-carbon double bond that can be an ethylenically unsaturated group or a vinyl group. Exemplary polymerizable groups include acrylate groups, methacrylate groups, ethacrylate groups, 2-phenyl acrylate groups, acrylamide groups, methacrylamide groups, itaconate groups, and styrene groups.

Polymers can be effectively derivatized in organic, polar, or anhydrous solvents, or solvent combinations to produce macromers. Generally, a solvent system is used that allows for polymer solubility and control over the derivatization with polymerizable groups. Polymerizable groups such as glycidyl acrylate can be added to polymers (including polysaccharides and polypeptides) in straightforward synthetic processes. In some aspects, the polymerizable group is present on the macromer at a molar ratio of 0.05 μmol or greater of polymerizable group (such as an acrylate group) per 1 mg of macromer. In some aspects the macromer is derivatized with polymerizable groups in amount in the range from about 0.05 μmol to about 2 μmol of polymerizable group (such as an acrylate group) per 1 mg of macromer.

Many polymers prepared from monomers with reactive oxygen-containing groups (such as oxides) have hydroxyl-containing terminal ends which can be reacted with a compound having a hydroxyl-reactive group and a polymerizable group to provide the macromer with polymerizable groups at its termini.

The first component can comprises a macromer based on a linear hydrophilic polymer, and can be formed from a biocompatible polymer that is hydrophilic. Exemplary polymers that that can be used to form the first component can be based on one or more of the following polymers: poly(vinylpyrrolidone) (PVP), poly(ethylene oxide) (PEO), poly(ethyloxazoline), poly(propylene oxide) (PPO), poly(meth)acrylamide (PAA) and poly(meth)acylic acid, poly(ethylene glycol) (PEG) (see, for example, U.S. Pat. Nos. 5,410,016, 5,626,863, 5,252,714, 5,739,208 and 5,672,662) PEG-PPO (copolymers of polyethylene glycol and polypropylene oxide), hydrophilic segmented urethanes (see, for example, U.S. Pat. Nos. 5,100,992 and 6,784,273), and polyvinyl alcohol (see, for example, U.S. Pat. Nos. 6,676,971 and 6,710,126).

In some aspects, the first component has a molecular weight in the range of 100 Da to 5000 Da, 100 Da to 10,000 Da, 100 Da to 20,000 Da, or 100 Da to 40,000 Da.

In some aspects, the macromer is formed from an oxyalkylene polymer, such as an ethylene glycol polymer or oligomer having the structure HO—(CH₂—CH₂—O)ₙ—H. As an example, the value of n ranges from about 3 to about 150 and the number average molecular weight (Mn) of the poly(ethylene glycol) ranges from about 100 Da to about 5000 Da, more typically ranging from about 200 Da to about 3500 Da.

An oxyalkylene polymer can be effectively derivatized to add polymerizable groups to produce oxyalkylene based macromers. Polymerizable groups such as glycidyl acrylate, glycidyl methacrylate, acrylic or methacrylic acid can be reacted with the terminal hydroxyl groups of these polymers to provide terminal polymerizable groups.

Some specific examples of alkylene oxide polymer-based macromers include, poly(propylene glycol)₅₄₀-diacrylate, poly(propylene glycol)₄₇₅-dimethacrylate, poly(propylene glycol)₉₀₀-diacrylate, poly(ethylene glycol)₂₅₀-diacrylate, poly(ethylene glycol)₅₇₅-diacrylate, poly(ethylene glycol)₅₅₀-dimethacrylate, poly(ethylene glycol)₇₅₀-dimethacrylate, poly(ethylene glycol)₇₀₀-diacrylate, and poly(ethylene glycol)₁₀₀₀-diacrylate, poly(ethylene glycol)₂₀₀₀diacrylate, poly(ethylene glycol)₁₀₀₀ monomethyl ether monomethacrylate, and poly(ethylene glycol)₅₀₀ monomethyl ether monomethacrylate. These types of alkylene oxide polymer-based macromers are available from Sigma-Aldrich (St. Louis, Mo.) or Polysciences (Warrington, Pa.).

The second component comprises the non-linear or branched compound comprising two or more hydrophilic polymeric portions and pendent reactive groups. In some cases, the second component includes pendent polymerizable groups pendent from the polymeric portions of the compound. In these cases, the compound can also be considered a macromeric compound, and can be used to form the swellable polymeric matrix in the same manner as the first component.

A “non-linear” or “branched” compound having polymeric portions refers to those having a structure different than a linear polymer (which is a polymer in which the molecules form long chains without branches or cross-linked structures). Such a compound can have multiple polymeric “arms” which are attached to a common linking portion of the compound. Non-linear or branched compounds are exemplified by, but not limited to, those having the following general structures:

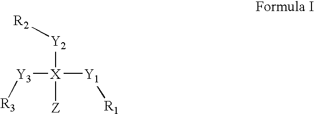

wherein X is a linking atom, such as one selected from C or S, or a linking structure, such a homo- or heterocyclic ring; to Y₁ to Y₃ are bridging groups, which can independently be, for example, —Cₙ—O—, wherein n is 0 or an integer of 1 or greater; R₁ to R₃ are independently hydrophilic polymeric portions, which can be the same or different, and have one or more pendent polymerizable groups; and Z is a non-polymeric group, such as a short chain alkyl group.

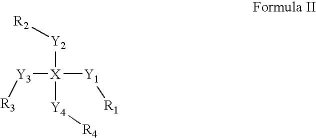

wherein X is a linking atom, such as one selected from C or S, or a linking structure, such a homo- or heterocyclic ring; to Y₁ to Y₄ are bridging groups, which can individually be, for example, —Cₙ—O—, wherein n is 0 or an integer of 1 or greater; and R₁ to R₄ independently hydrophilic polymeric portions, which can be the same or different, and have one or more pendent polymerizable groups.

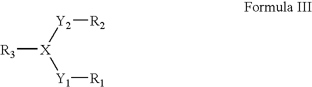

wherein X is a linking atom or group, such as one selected from N, C—H, or S—H, or a linking structure, such a homo- or heterocyclic ring; to Y₁ and Y₂ are bridging groups, which can individually be, for example, —Cₙ—O—, wherein n is 0 or an integer of 1 or greater; and R₁ to R₃ independently hydrophilic polymeric portions, which can be the same or different, and have one or more pendent polymerizable groups.

In many aspects the branched compounds have one polymerizable group per polymeric branched portion (R) of the compound. In many aspects the polymerizable groups are located at the termini of the polymeric portions R.

The second compound can be prepared from a polyol, such as a low molecular weight polyol (for example, a polyol having a molecular weight of 200 Da or less). In some aspects the second compound can be derived from a triol, a tetraol, or other multifunctional alcohol. Exemplary polyol derivatives include derivatives of pentaerythritol, trimethylolpropane, and glycerol.

The polymeric portions of the second compound can be selected from PVP, PEO, poly(ethyloxazoline), PPO, PAA and poly(meth)acylic acid, PEG, and PEG-PPO, hydrophilic segmented urethanes, and polyvinyl alcohol, such as those described herein.

In some aspects, the second component comprises one or more polymeric portions that is or are an oxyalkylene polymer, such as an ethylene glycol polymer.

For example, the preparation of a PEG-triacrylate macromer (trimethylolpropane ethoxylate (20/3 EO/OH) triacrylate macromer), which can be used as the second component, is described in Example 5 of commonly assigned U.S. Patent Application Publication No. 2004/0202774A1 (Chudzik, et al.).

The non-linear or branched compound can derived from a polyol, such as one having a molecular weight of less than 200 Da.

In some aspects, the second component has a molecular weight in the range of about 300 Da to about 20 kDa, or more specifically in the range of about 500 Da to about 2500 Da.

A composition can be prepared containing the first compound (the macromer based on a linear hydrophilic polymer) and the second compound (the non-linear or branched compound comprising two or more hydrophilic polymeric portions and pendent reactive groups) at concentrations sufficient to form the polymeric matrix that can be swollen to a durable hydrogel.

The composition including the first and second components can have a viscosity that is suitable for the type of matrix-forming process performed. In order to prepare a composition, the first and second components (and any other component), can be dissolved or suspended in a suitable polar liquid. Exemplary polar liquids include alcohol or water. Combinations of polar solvents can also be used. In some aspects, the viscosity of the composition is in the range of about 5 to 200 cP (at about 25° C.).

In some aspects, the matrix-forming composition includes the first component at a concentration of about 5% wt solids or greater and a second component at a concentration of about 5% wt solids or greater. An exemplary range for the first component is from about 2% wt solids to about 40% wt solids, and more specifically from about 5% wt solids to about 30% wt solids. An exemplary range for the second component is from about 2% wt solids to about 40% wt solids, and more specifically from about 5% wt solids to about 30% wt solids.

Another way of describing the matrix-forming composition is by reference to the total amount solids of the first and second component in the composition, or the total amount of polymerizable material in the composition. For example, in some aspects, the matrix-forming composition includes the first component and second component, and any other optional polymerizable component, at a concentration of about 10% wt solids or greater, or the first component and second component, and any other optional polymerizable component, at a concentration in the range of about 10% wt solids to about 60% wt solids.

In some aspects the composition or matrix has an amount the first polymer-containing segment (linear component) and the second polymer-containing segment (branched component) at a weight ratio in the range of 100:1 to 1:100, respectively. In some aspects the composition or matrix has an amount the first polymer-containing segment (linear component) and the second polymer-containing segment (branched component) at a weight ratio in the range of 50:1 to 1:10, respectively.

In some aspects, the composition includes an initiator that is capable of promoting the formation of a reactive species from a polymerizable group. For example, the initiator can promote a free radical reaction of hydrophilic polymer having pendent polymerizable groups. In one embodiment the initiator is a compound that includes a photoreactive group (photoinitiator). For example, the photoreactive group can include an aryl ketone photogroup selected from acetophenone, benzophenone, anthraquinone, anthrone, anthrone-like heterocycles, and derivatives thereof.

In some aspects the photoinitiator includes one or more charged groups. The presence of charged groups can increase the solubility of the photoinitiator (which can contain photoreactive groups such as aryl ketones) in an aqueous system. Suitable charged groups include, for example, salts of organic acids, such as sulfonate, phosphonate, carboxylate, and the like, and onium groups, such as quaternary ammonium, sulfonium, phosphonium, protonated amine, and the like. According to this embodiment, a suitable photoinitiator can include, for example, one or more aryl ketone photogroups selected from acetophenone, benzophenone, anthraquinone, anthrone, anthrone-like heterocycles, and derivatives thereof; and one or more charged groups. Examples of these types of water-soluble photoinitiators have been described in U.S. Pat. No. 6,278,018.

Water-soluble polymerization initiators can be used at a concentration sufficient to initiate polymerization of the first and second components and formation of the matrix. For example, a water-soluble photo-initiator as described herein can be used at a concentration of about 0.5 mg/mL or greater. In some modes of practice, the photo-initiator is used at a concentration about 1.0 mg/mL along with the matrix-forming components.

Thermally reactive initiators can also be used to promote the polymerization of hydrophilic polymers having pendent coupling groups. Examples of thermally reactive initiators include 4,4′ azobis(4-cyanopentanoic acid), 2,2-azobis[2-(2-imidazolin-2-yl)propane]dihydrochloride, and analogs of benzoyl peroxide. Redox initiators can also be used to promote the polymerization of the hydrophilic polymers having pendent coupling groups. In general, combinations of organic and inorganic oxidizers, and organic and inorganic reducing agents are used to generate radicals for polymerization. A description of redox initiation can be found in Principles of Polymerization, 2^{nd} Edition, Odian G., John Wiley and Sons, pgs 201-204, (1981).

Alternatively, formation of the swellable polymeric matrix can be caused by the combination of an oxidizing agent/reducing agent pair, a “redox pair,” in the presence of the matrix-forming material.

The oxidizing agent can be selected from inorganic or organic oxidizing agents, including enzymes; the reducing agent can be selected from inorganic or organic reducing agents, including enzymes. Exemplary oxidizing agents include peroxides, including hydrogen peroxide, metal oxides, and oxidases, such as glucose oxidase. Exemplary reducing agents include salts and derivatives of electropositive elemental metals such as Li, Na, Mg, Fe, Zn, Al, and reductases. In one aspect, the reducing agent is present in the composition at a concentration of 2.5 mM or greater when mixed with the oxidizing agent. Other reagents, such as metal or ammonium salts of persulfate, can be present in the composition to promote polymerization of the matrix-forming composition.

The redox pair can be combined in the presence of the matrix-forming material in any suitable manner. For example, a first composition containing the first component and the oxidizing agent, and a second composition including the reducing agent and the second component, can be prepared. Upon mixing of the first and second compositions polymerization commences and the swellable polymeric matrix begins to form.

The matrix-forming composition can also include one or more other ancillary reagent(s) that help promote formation of the matrix. These reagents can include polymerization co-initiators, reducing agents, and/or polymerization accelerants known in the art. These ancillary agents can be included in the composition at any useful concentration.

Exemplary co-initiators include organic peroxides, such as those that are derivatives of hydrogen peroxides (H₂O₂) in which one or both of the hydrogen atoms are replaced by an organic group. Organic peroxides contain the —O—O— bond within the molecular structure, and the chemical properties of the peroxides originate from this bond. The peroxide polymerization co-initiator can be a stable organic peroxide, such as an alkyl hydroperoxide. Exemplary alkyl hydroperoxides include t-butyl hydroperoxide, p-diisopropylbenzene peroxide, cumene hydroperoxide, acetyl peroxide, t-amyl hydrogen peroxide, and cumyl hydrogen peroxide.

Other polymerization co-initiators include azo compounds such as 2-azobis(isobutyronitrile), ammonium persulfate, and potassium persulfate.

The matrix-forming composition can include a reducing agent such as a tertiary amine. In many cases the reducing agent, such as a tertiary amine, can improve free radical generation. Examples of the amine compound include primary amines such as n-butylamine; secondary amines such as diphenylamine; aliphatic tertiary amines such as triethylamine; and aromatic tertiary amines such as p-dimethylaminobenzoic acid.

In other aspects of the invention, in addition to these components, the composition used to form the swellable polymeric matrix can include one or more polymerization accelerator(s). A polymerization accelerator such as n-vinyl pyrrolidone can be used. In some aspects a polymerization accelerator having a biocompatible functional group (e.g., a biocompatible polymerization accelerator) is included in the composition of the present invention. The biocompatible polymerization accelerator can also include an N-vinyl group such as N-vinyl amide group. Biocompatible polymerization accelerators are described in commonly assigned U.S. Patent Application Publication No. 2005/0112086.

In some aspects of the invention, the swellable polymeric matrix is formed in association with a medical device. For example, the matrix can be formed as an overmold or a coating in association with a part of, or the entire device.

A “coating” refers to one or more layers of matrix material, formed by applying the matrix forming materials to all or a portion of a surface of an article by conventional coating techniques.

An “overmold” refers to matrix material formed in association with all or a portion of a surface of an article. An overmold of matrix material is generally thicker than a coating, and typically formed using a molding process rather than a coating process.

A “medical device” refers to an article used in a medical procedure. Typically, the matrix is formed on the surface of an implantable medical device. From a structural standpoint, the implantable medical device may be a simple article, such as a rod, pellet, sphere, or wire, on which the swellable matrix can be formed. The implantable medical device can also have a more complex structure or geometry, as would be found in an intralumenal prosthesis, such as a stent.

An implantable device having a swellable polymeric matrix (formed using the hydrogel-forming materials of the invention), or a portion thereof, can be configured to be placed within the vasculature (an implantable vascular device), such as in an artery, vein, fistula, or aneurysm. In some cases the implantable device is an occlusion device selected from vascular occlusion coils, wires, or strings that can be inserted into aneurysms. Some specific vascular occlusion devices include detachable embolization coils. In some cases the implantable device is a stent.

Alternatively, the implantable device, or a portion thereof, can be configured to be placed within other body lumens, such as the fallopian tubes, bile ducts, etc. For example, the implantable device can be placed at one or more portions of the urogenital system. Some exemplary implantable urogenital devices are used for birth control, for example, fabric-containing occlusive coils which are inserted into the fallopian tubes by hysteroscopy (Conceptus, Mountain View, Calif.).

Other medical articles on which the swellable polymeric matrix can be formed include, but are not limited to, small diameter grafts, abdominal aortic aneurysm grafts; wound dressings and wound management devices; hemostatic barriers; mesh and hernia plugs; patches, including uterine bleeding patches, atrial septic defect (ASD) patches, patent foramen ovale (PFO) patches, ventricular septal defect (VSD) patches, and other generic cardiac patches; ASD, PFO, and VSD closures; percutaneous closure devices; birth control devices; breast implants; orthopedic devices such as orthopedic joint implants, bone repair/augmentation devices, cartilage repair devices; urological devices and urethral devices such as urological implants, and bladder devices.

Implantable medical devices can be prepared from metals such as platinum, gold, or tungsten, although other metals such as rhenium, palladium, rhodium, ruthenium, titanium, nickel, and alloys of these metals, such as stainless steel, titanium/nickel, and nitinol alloys, can be used.

The surface of metal-containing medical devices can be pretreated (for example, with a Parylene™-containing coating composition) in order to alter the surface properties of the biomaterial, when desired. Metal surfaces can also be treated with silane reagents, such as hydroxy- or chloro-silanes.

Implantable medical devices can also be partially or entirely fabricated from a plastic polymer. In this regard, the swellable polymeric matrix can be formed on a plastic surface. Plastic polymers include those formed of synthetic polymers, including oligomers, homopolymers, and copolymers resulting from either addition or condensation polymerizations. Examples of suitable addition polymers include, but are not limited to, acrylics such as those polymerized from methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide; vinyls such as ethylene, propylene, vinyl chloride, vinyl acetate, vinyl pyrrolidone, vinylidene difluoride, and styrene. Examples of condensation polymers include, but are not limited to, nylons such as polycaprolactam, polylauryl lactam, polyhexamethylene adipamide, and polyhexamethylene dodecanediamide, and also polyurethanes, polycarbonates, polyamides, polysulfones, poly(ethylene terephthalate), polydimethylsiloxanes, and polyetherketone.

A medical device with a swellable polymeric matrix “overmold” can be formed in a process using a mold, a composition comprising the matrix-forming material, and a medical device. The medical device can be placed in a portion of the mold so the composition can be placed in contact with all or a portion of the surface of the device. For example, a device in the shape of a rod or coil is fixtured in a mold so that that composition can be in contact with the entire surface of the device. The composition can then be added to mold and treated to promote matrix formation. In some cases, the mold is made of a material that allows UV light to pass through it, and the composition can include a photo-initiator, which is activated by the UV and causes matrix formation.

In another exemplary mode of preparation, a matrix overmold can be formed by adding the composition to the mold and then partially polymerizing the matrix so the composition increases in viscosity. The medical device can then be placed in the partially polymerized composition, and due to its increased viscosity, suspends the device within the composition as desired. The composition can then be fully polymerized to solidify the materials of the composition, which forms the swellable polymeric matrix as an overmold on the device. After the swellable polymeric matrix forms as an overmold, the device can be removed from the mold.

The weight of the polymeric matrix can be a substantial percentage of the weight of the overall device. When the matrix is in a partially hydrated for fully hydrated state, it can have a weight that is substantially greater than the device which it overmolds.

The overmold can be formed on any desired medical device and can dimensions suitable for occluding a target site in the body. The swellable polymeric matrix in an overmold is typically thicker than the matrix of a coating, which can provide advantages for occlusion of a target site.

In some aspects, the matrix has a thickness in the range of about 50 μm to about 500 μm, and more specifically in the range of about 100 μm to about 300 μm. The matrix can then be dried to have a thickness in the range of about 25 μm to about 400 μm, respectively, and more specifically in the range of about 75 μm to about 250 μm, respectively. The matrix can be hydrated (e.g., in vivo), which can swell the matrix to a thickness in the range of about 100 μm to about 2500 μm, respectively, and more specifically in the range of about 750 μm to about 1500 μm, respectively.

As a specific example, in the case of a fallopian tube occlusion coil having a diameter of about 0.5 mm, a swellable polymeric matrix in the form of an overmold is formed on the coil. The overmold has a thickness in the range of about 100 μm to about 450 μm, or about 100 μm to about 300 μm in a dried state. During and/or after delivery of the article to the fallopian tube, the coating swells to have a thickness in the range of about 750 μm to about 1500 μm, causing occlusion of the fallopian tube and prevention of fertilization.

A device with an overmolding can be delivered to a target site in the body, where it hydrates to a hydrogel within the target site. Delivery of the device can be performed using a catheter and/or other guide instruments, such as guidewires.

The swellable polymeric matrix can become hydrated in a relatively short period of time, such as period of time in the range of about 30 minutes to about 2 hours, or about 1 hour. Swelling of the polymeric matrix can be monitored to determine if the hydrogel occludes the target site as desired.

In another aspect, the swellable polymeric matrix is in the form of a coating on a medical device. A matrix coating that includes the first and second compounds can be formed various ways.

In one mode of practice, a composition including the first and second compounds is dip-coated onto the surface of the substrate to form a coating. The composition on the surface can then be treated to cause matrix formation. For example, a composition including the first and second compounds, and a photoactivatable polymerization initiator is dipcoated on the surface of a device. During and/or after the dip-coating step, the applied material can be irradiated to promote polymerization of the first and second components, and matrix formation.

Other techniques, such as brushing or spraying the composition can be used to form the coating. The method of spray coating can be performed by spraying the composition on the surface the device, and then treating the composition to form the coating.

In another aspect of the invention, the (first) linear hydrophilic polymer, and a (second) non-linear or branched compound comprising two or more hydrophilic polymeric portions, each having pendent reactive groups, is used to form a polymeric matrix article which is capable of swelling to a hydrogel. A device such as one used in an overmolding process is not used as a portion of the article, and the swellable matrix itself forms the implantable article.

Such an implantable matrix article can have a simple or a complex geometry. A simple geometry is exemplified by a device that is in the form of a filament (e.g., threads, strings, rods, etc.). A matrix article with a simple geometry can be prepared by various methods. One method for forming the matrix article uses the same process as used to form the overmolded device, but does not include a device within the mold. Again, the mold can be, for example, a piece of tubing which has an inner area corresponding to the first configuration of the body member. The composition can then be injected into the tubing to fill the tubing. The composition in the tubing can then be treated to activate the polymerization initiator (such as by photo-initiated polymerization). Polymerization promotes crosslinking of the first and second components (and any other optional polymerizable material) and establishes a polymeric matrix in the configuration of the mold.

In many cases, the matrix article can be used in the same way that the overmolded device is used.

The polymerizable materials of the present invention can also be used for the formation of an in situ polymerized mass at a target site in the body. Generally, a composition that includes the first and second components can be delivered to or applied to a target site, and then the composition is treated to promote polymerization and formation of the swellable polymeric matrix. In some cases, two separate solutions (for example, each having a member of a redox pair) are delivered to, and mixed at a target site in situ. The mixing of the solutions causes polymerization and formation of the swellable polymeric matrix at the target site. The matrix formed at the target site hydrates to occlude the target site.

In some modes of practice, a composition containing the polymerizable materials can be passed through a small gauge delivery conduit to place the composition at a target site. Polymerization and matrix formation can occur in situ. Delivering a polymerizable composition to the target site (such as a neuroaneurysm) can be performed using a microcatheter, for example, one having a diameter of less than 2.3 french.

The swellable polymeric matrices of the present invention can also include a bioactive agent, releasable from, and/or stable in the hydrogel. Examples of bioactive agents that can be included in the hydrogel include: ACE inhibitors, actin inhibitors, analgesics, anesthetics, anti-hypertensives, anti polymerases, antisecretory agents, anti-AIDS substances, antibiotics, anti-cancer substances, anti-cholinergics, anti-coagulants, anti-convulsants, anti-depressants, anti-emetics, antifungals, anti-glaucoma solutes, antihistamines, antihypertensive agents, anti-inflammatory agents (such as NSAIDs), anti metabolites, antimitotics, antioxidizing agents, anti-parasite and/or anti-Parkinson substances, antiproliferatives (including antiangiogenesis agents), anti-protozoal solutes, anti-psychotic substances, anti-pyretics, antiseptics, anti-spasmodics, antiviral agents, calcium channel blockers, cell response modifiers, chelators, chemotherapeutic agents, dopamine agonists, extracellular matrix components, fibrinolytic agents, free radical scavengers, growth hormone antagonists, hypnotics, immunosuppressive agents, immunotoxins, inhibitors of surface glycoprotein receptors, microtubule inhibitors, miotics, muscle contractants, muscle relaxants, neurotoxins, neurotransmitters, polynucleotides and derivatives thereof, opioids, photodynamic therapy agents, prostaglandins, remodeling inhibitors, statins, steroids, thrombolytic agents, tranquilizers, vasodilators, and vasospasm inhibitors. One or more bioactive agents can be present in the polymeric matrix in an amount sufficient to provide a biological response.

In some aspects, the swellable polymeric matrix can also include a pro-fibrotic agent. A pro-fibrotic agent can promote a rapid and localized fibrotic response in the vicinity of the hydrogel. This can lead to the accumulation of clotting factors and formation of a fibrin clot in association with the hydrogel. In some aspects the pro-fibrotic agent is a polymer. The polymer can be based on a natural polymer, such as collagen, or a synthetic polymer.

The swellable durable polymeric matrix can also include an imaging material. Imaging materials can facilitate visualization of the polymeric matrix one implanted or formed in the body. Medical imaging materials are well known. Exemplary imaging materials include paramagnetic material, such as nanoparticular iron oxide, Gd, or Mn, a radioisotope, and non-toxic radio-opaque markers (for example, cage barium sulfate and bismuth trioxide). Radiopacifiers (such as radio opaque materials) can be included in a composition used to make the matrix. The degree of radiopacity contrast can be altered by controlling the concentration of the radiopacifier within the matrix. Common radio opaque materials include barium sulfate, bismuth subcarbonate, and zirconium dioxide. Other radio opaque materials include cadmium, tungsten, gold, tantalum, bismuth, platinum, iridium, and rhodium

Paramagnetic resonance imaging, ultrasonic imaging, x-ray means, fluoroscopy, or other suitable detection techniques can detect the swellable or swollen matrices that include these materials. As another example, microparticles that contain a vapor phase chemical can be included in the matrix and used for ultrasonic imaging. Useful vapor phase chemicals include perfluorohydrocarbons, such as perfluoropentane and perfluorohexane, which are described in U.S. Pat. No. 5,558,854 (Issued 24 Sep. 1996); other vapor phase chemicals useful for ultrasonic imaging can be found in U.S. Pat. No. 6,261,537 (Issued 17 Jul. 2001).

Testing can be carried out to determine mechanical properties of the hydrogel. Dynamic mechanical thermal testing can provide information on the viscoelastic and rheological properties of the hydrogel by measuring its mechanical response as it is deformed under stress. Measurements can include determinations of compressive modulus, and shear modulus. Key viscoeslatic parameters (including compressive modulus and sheer modulus) can be measured in oscillation as a function of stress, strain, frequency, temperature, or time. Commercially available rheometers (for example, available from (TA Instruments, New Castle, Del.) can be used to make these measurements. The testing of hydrogels for mechanical properties is also described in Anseth et al. (1996) Mechanical properties of hydrogels and their experimental determination, Biomaterials, 17:1647.

The hydrogel can be measured to determine its complex dynamic modulus (G*): G*=G′+iG″=σ*/γ*, where G′ is the real (elastic or storage) modulus, and G″ is the imaginary (viscous or loss) modulus, these definitions are applicable to testing in the shear mode, where G refers to the shear modulus, σ to the shear stress, and γ to the shear strain.

The hydrogels of the present invention can have a compressive modulus, such as greater than 500 kPa, or greater than 2000 kPa.

The hydrogel can also be measured for its swelling (or osmotic) pressure. Commercially available texture analyzers (for example, available from Stable Micro Systems; distributed by Texture Technologies Corp; Scarsdale, N.Y.) can be used to make these measurements. Texture analyzers can allow measurement of force and distance in tension or compression.

In some modes of practice, hydrogels having swelling pressures in the range of about 10 kPa (about 100 g/cm²)-to about 750 kPa (about 7600 g/cm²), or about 10 kPa (100 g/cm²) to 196 kPa (2000 g/cm²) are used. In other words, the matrix is capable of exerting a swelling force in these ranges upon hydration from a dehydrated or partially hydrated form.

In some formations, the polymeric matrix is capable of swelling in water to a weight in the range of 1.5 to 10 times its weight in a dehydrated form. In some formations, the polymeric matrix is capable of swelling in water to a size in the range of about 150% to about 300%, about 150% to about 250% its size in a dehydrated form.

### EXAMPLE 1

### Ultra Violet Cross-linking of Poly(Ethylene Glycol) Diacrylate Compounds

Into an amber vial, 4,5-bis(4-benzoylphenylmethyleneoxy)benzene-1,3-disulfonic acid (5 mg)(DBDS), prepared as described in U.S. Pat. No. 6,278,018 (Example 1) and commercially available from SurModics, Inc. (Eden Prairie, Minn.) was weighed and dissolved into deionized water at a concentration of 1 mg/mL. The solution was vortexed and sonicated to ensure a homogeneous mixture. The poly(ethylene glycol) diacrylate (Sigma-Aldrich, St. Louis, Mo.; Avg. MW=700, cat. #455008) or the poly(ethylene glycol) dimethacrylate (Monomer-Polymer and Dajac Laboratories, Inc., Feasterville, Pa.; 9362 Polyethylene Glycol 1000 Diacrylate) was weighed and dissolved into the DBDS solution at 300 mg/mL. A trimethylolpropane ethoxylate (20/3 EO/OH) triacrylate macromer “PEG-triacrylate macromer” (PEG-TA; the preparation of is described in Example 5 of commonly assigned U.S. Patent Application Publication No. 2004/0202774A1 (Chudzik, et al.)) was weighed and dissolved into the DBDS solution at a concentration of 300 mg/mL. Since the solubility of these reagents in water is very high, a large range of concentrations can be made.

Into a 9 mm wide×4 mm deep diameter Teflon well, 100 μL of the PEG-triacrylate macromer and 130 μL of poly(ethylene glycol) diacrylate were pipetted and mixed gently. A Dymax 2000-EC series UV floodlamp with 400 watt metal halide bulb was used to initiate cross-linking of the polymers. The sample was placed in the chamber 20 cm from the light source for two minutes to ensure a complete photochemical reaction.

The physical properties of the gels were determined by compression force testing and swellability testing. A TAXT2 texture analyzer with 5 mm diameter ball probe was used to determined compression strength. The procedure used a test speed of 0.5 mm/sec and a trigger force of 4 g. The probe compressed to 25% of the depth of the material as compared to the calibration depth. The resulting force of the gel was 509.7 g. The polymer was dried fully and an initial weight was taken. It was then placed into a vial with 400 μL of deionized water and allowed to swell for 24 hours. A final weight was taken and the gel showed swelling of 367%.

### EXAMPLE 2

### Radiopaque Swellable Matrices Including Poly(Ethylene Glycol) Diacrylate and Trimethylolpropane Ethoxylate Triacrylate

Into an amber vial, the DBDS (see Example 1) was weighed and dissolved into deionized water at a concentration of 1 mg/mL. The solution was vortexed and sonicated to ensure a homogeneous mixture. Poly(ethylene glycol) diacrylate (Sigma-Aldrich, St. Louis, Mo.) was weighed and dissolved into the initiator solution at a concentration of 300 mg/mL. Photo-polyacrylamide (see U.S. Pat. No. 6,007,833, Examples 1 & 2; also, SurModics, Inc., Eden Prairie, Minn. (PA05)) was weighed and dissolved into the photoinitiator solution at a concentration of 80 mg/mL. Trimethylolpropane ethoxylate triacrylate (Example 1) was weighed and dissolved into the photoinitiator solution at a concentration of 100 mg/mL. A 55:40:5 ratio (v/v) of the three solutions was prepared. To this solution a radiopaque agent, barium sulfate, was added at 7.5% of the total weight of the solution. The complete solution was vortexed to ensure complete mixing of the reagents. The pre-crosslinked solution was pipetted into silicon tubing (HelixMark, Carpinteria, Calif.) with an inner diameter of 1.98 mm. To crosslink the material, the tubing was placed into a Blue Wave UV lamp for 90 seconds.

### Swelling

The filament was cut into 5 mm sections and removed from the tubing as a radiopaque polymer. The filament was fully dried in a dry chamber for 18 hours. The diameter of the polymer filament was measured using a Leica MZ12₅ stereomicroscope with Techniquip™ lighting and ImagePro™-Plus software version 6.1. Next, the filament was placed into a glass vial with deionized water and hydrated at 25° C. The stereomicroscope was again used to measure the diameter of the filament to determine swelling. The final measurement determined the swelling to be 190%.

### EXAMPLES 3-12

### Swellable Matrices Including Poly(Ethylene Glycol) Diacrylate and Trimethylolpropane Ethoxylate Triacrylate Polymers

Swellable polymeric matrices were prepared according to the method of Example 1 with the following changes in reagents, according to Table 1. Poly(ethylene glycol)₁₀₀₀ diacrylate (PEG-DA₁₀₀₀), poly(ethylene glycol)₂₀₀₀ diacrylate (PEG-DA₂₀₀₀), poly(ethylene glycol)₇₀₀ dimethacrylate (PEGDMA₇₀₀), poly(ethylene glycol)₁₀₀₀ monomethyl ether monomethacrylate (PEG-MEMA₁₀₀₀), and poly(ethylene glycol)₅₀₀ monomethyl ether monomethacrylate (PEG-MEMA₅₀₀) are available from Sigma-Aldrich (St. Louis, Mo.) or Polysciences (Warrington, Pa.).

TABLE 1

| Example | Branched Reagent | Linear Reagent | | Other | | Ratio | Swelling |
|---|---|---|---|---|---|---|---|
| 3 | PEG-TA (300 mg/mL) | PEGDA₇₀₀ | (300 mg/mL) | — | | 1:3 | 151% |
| 4 | PEG-TA (300 mg/mL) | PEGDA₇₀₀ | (300 mg/mL) | — | | 1:1 | 132% |
| 5 | PEG-TA (300 mg/mL) | PEGDA₇₀₀ | (300 mg/mL) | PEGDA₂₀₀₀ | (300 mg/mL) | 1:1:1 | 154% |
| 6 | PEG-TA (300 mg/mL) | PEGDA₁₀₀₀ | (300 mg/mL) | — | | 1:1 | 167% |
| 7 | PEG-TA (200 mg/mL) | PEGDA₁₀₀₀ | (300 mg/mL) | — | | 1:4 | 190% |
| 8 | PEG-TA (200 mg/mL) | PEGDMA₇₅₀ | (150 mg/mL) | PEGMEMA₁₀₀₀ | (150 mg/mL) | 1.5:1:2 | 186% |
| 9 | PEG-TA (125 mg/mL) | — | | PEGMEMA₅₀₀ | (125 mg/mL) | 1:4 | α |
| 10 | PEG-TA (125 mg/mL) | PEGDMA₇₅₀ | (125 mg/mL) | PEGMEMA₅₀₀ | (125 mg/mL) | 1:1:2 | 262% |
| 11 | PEG-TA (125 mg/mL) | PEGDMA₇₅₀ | (125 mg/mL) | PEGMEMA₅₀₀ | (125 mg/mL) | 1:5:5 | 236% |

| | | | | | | |
|---|---|---|---|---|---|---|
| 12 | — | PEGDA₂₀₀₀ | (150 mg/mL) | — | 1 | α |

Note:
All reagents were in a 1 mg/mL solution of photoinitiator (Ex. 2) and cured for 2 minutes under UV light
α Matrix did not form

What is claimed is:

1. A biocompatible swellable or swollen polymeric matrix comprising first and second polymer-containing segments crosslinked via polymerized groups, wherein the first polymer-containing segment has a linear structure comprising a hydrophilic polymer portion and the second polymer-containing segment has a branched structure comprising hydrophilic polymer portions.

2. The biocompatible swellable or swollen polymeric matrix of claim 1 wherein the second polymer-containing segment having a branched structure comprises oxyalkylene polymer portions.

3. The biocompatible swellable or swollen polymeric matrix of claim 1 wherein the second polymer-containing segment has a molecular weight in the range of 300 Da to 20 kDa.

4. The biocompatible swellable or swollen polymeric matrix of claim 1 wherein the alkylene oxide polymer portions of the second polymer-containing segment independently have a molecular weight in the range of 500 Da to 2500 Da.

5. The biocompatible swellable or swollen polymeric matrix of claim 1 wherein the second polymer-containing segment has a branched structure with three alkylene oxide polymer portions.

6. The biocompatible swellable or swollen polymeric matrix of claim 1 wherein second polymer-containing segment having a branched structure comprises poly(ethylene glycol) portions.

7. The biocompatible swellable or swollen polymeric matrix of claim 1 wherein the second polymer-containing segment has a structure selected from the group consisting of: 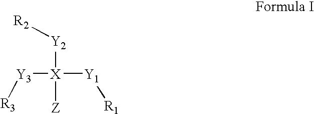
wherein X is C or S, or a homo- or heterocyclic ring; Y₁, Y₂, and Y₃ are independently, —Cₙ—O—, wherein n is 0 or an integer of 1 or greater; R₁, R₂, and R₃, are independently hydrophilic polymeric portions, which can be the same or different, and R₁, R₂, and R₃ independently have one or more pendent polymerized group(s); and Z is a non-polymeric group; 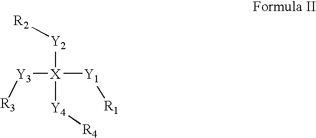
wherein X is C or S, or a homo- or heterocyclic ring; Y₁, Y₂, Y₃, and Y₄ are independently, —Cₙ—O—, wherein n is 0 or an integer of 1 or greater; R₁, R₂, R₃, and R₄, are independently hydrophilic polymeric portions, which can be the same or different, and R₁, R₂, R₃, and R₄ independently have one or more pendent polymerized group(s); and 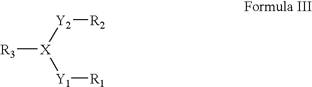
wherein X is selected from N, C—H, or S—H, or a homo- or heterocyclic ring; Y₁ and Y₂ are —Cₙ—O— wherein n is 0 or an integer of 1 or greater; and R₁, R₂, and R₃, are independently hydrophilic polymeric portions, which can be the same or different, and R₁, R₂, and R₃ independently have one or more pendent polymerized group(s).

8. The biocompatible swellable or swollen polymeric matrix of claim 1 wherein the first polymer-containing segment having a linear structure comprises a polymer selected from the group consisting of poly(vinylpyrrolidone) (PVP), poly(ethylene oxide) (PEO), poly(ethyloxazoline), poly(propylene oxide) (PPO), poly(meth)acrylamide (PAA) and poly(meth)acylic acid, poly(ethylene glycol) (PEG), copolymers of polyethylene glycol and polypropylene oxide (PEG-PPO), hydrophilic segmented urethanes, and polyvinyl alcohol.

9. The biocompatible swellable or swollen polymeric matrix of claim 1 wherein the first polymer-containing segment having a linear structure comprises an oxyalkylene polymer.

10. The biocompatible swellable or swollen polymeric matrix of claim 1 wherein the first polymer-containing segment having a linear structure comprise a hydrophilic polymer portion having a molecular weight of in the range of 100 Da to 5000 Da.

11. The biocompatible swellable or swollen polymeric matrix of claim 1 wherein the first polymer-containing segment and the second polymer-containing segment are present in the matrix at a weight ratio in the range of 100:1 to 1:100, respectively.

12. The biocompatible swellable or swollen polymeric matrix of claim 11 wherein the first polymer-containing segment and the second polymer-containing segment are present in the matrix at a weight ratio in the range of 50:1 to 1:10, respectively.

13. The biocompatible swellable polymeric matrix of claim 1 which is capable of swelling in water to a weight in the range of 1.5 to 10 times a weight of the matrix in a dehydrated form.

14. The biocompatible swellable polymeric matrix of claim 1 which exerts a swelling force in the range of 100 g/cm² to 2000 g/cm² upon hydration from a dehydrated form.

15. The biocompatible swellable polymeric matrix of claim 1 which is capable of swelling in water to a size in the range of about 150% to about 300% its size in a dehydrated form.

16. The biocompatible swellable or swollen polymeric matrix of claim 1 which is associated with an implantable medical device.

17. The biocompatible swellable or swollen polymeric matrix of claim 1 which is in the form of an overcoat on the implantable medical device.

18. The biocompatible swellable or swollen polymeric matrix of claim 1 comprising a radioopaque agent.

19. The biocompatible swellable or swollen polymeric matrix of claim 1 further comprising a third segment comprising a hydrophilic polymer comprising pendent reacted photogroups.

20. A method for forming a biocompatible swellable or swollen polymeric matrix comprising steps of:
(a) providing a composition comprising
(i) a first compound having a linear structure comprising a hydrophilic polymer portion and a pendent polymerizable group, and
(ii) a second compound having a branched structure comprising oxyalkylene polymer portions and pendent polymerizable groups, and
(b) activating the polymerizable groups to cause crosslinking of the first and second compounds and matrix formation.

21. The method of claim 20 wherein the composition comprises the second compound in an amount of 5% wt solids or greater.

22. The method of claim 20 wherein the composition comprises the first compound in an amount in the range of 2% wt solids to 40% wt solids.

23. The method of claim 20 where, in step (a) the composition is provided to a target location on a subject, and in step (b) the polymerizable groups are activated to cause crosslinking of the first and second compounds and in situ matrix formation.

24. A method for treating a subject comprising a step of placing a biocompatible swellable or swollen polymeric matrix according to claim 1 at a target location in a subject.

25. The method of claim 24 resulting in a swollen polymeric matrix which occludes the target location.
